(19) United States
(12) Patent Application Publication
(10) Pub. No.: US 2006/0069044 A1
(43) Pub. Date: Mar. 30, 2006

(54) Modified glycosaminoglycans, pharmaceutical compositions and methods for oral delivery thereof

(76) Inventors: Sigmund E. Lasker, New York, NY (US); Biswajit Lahiri, Yorktown Heights, NY (US)

Correspondence Address: COOPER & DUNHAM, LLP, 1185 AVENUE OF THE AMERICAS, NEW YORK, NY, 10036, US

(21) Appl. No.: 11/220,078

(22) Filed: Sep. 6, 2005

Related U.S. Application Data
(60) Provisional application No. 60/608,830, filed on Sep. 10, 2004.

(51) Int. Cl. A61K 31/727

(52) U.S. Cl. 514/26; 514/54; 514/56

(57) ABSTRACT

This invention provides a composition comprising a depolymerized glycosaminoglycan or derivative thereof covalently linked to a bile acid. This invention also provides a pharmaceutical composition comprising a depolymerized glycosaminoglycan or derivative thereof covalently linked to a bile acid and a pharmaceutically acceptable carrier. Finally, this invention provides a method for orally delivering heparin to a subject comprising administering to the subject a pharmaceutically effective amount of a pharmaceutical composition comprising a glycosaminoglycan or derivative thereof covalently linked to a bile acid and (b) a pharmaceutically acceptable carrier.

This application claims priority of U.S. Provisional Patent Application No. 60/608,830, filed on Sep. 10, 2004, the contents of which are incorporated herein by reference.

### BACKGROUND OF THE INVENTION

The Glycosaminoglycans (GAG's) are copolymers of uronic acid and amino hexose with a sequence that has unique biological activity. When prepared from natural sources, this family of heterogeneous macromolecules has sequences that bind specific proteins providing them with various biological activities. Some of the interactions that are of therapeutic utility are between the serine proteases generally and in particular the blood protein antithrombin. This interaction is dependent on special structural sequences that are present in the depolymerized natural product.

The multiplicity of commercially available preparations of heparins are manufactured by diverse processes with variable starting materials yielding products with unique structural components and different biological activities. The commercial products called heparins are thus generally undefined tissue derived panoply of long chain polysaccharides. The structural variability may be a result of the extraction process, the source and condition of the starting materials as well as the collection methods. The resulting biological activities of the product are influenced by these variables.

Heparins are common GAG's made up mainly of D-glucosamine and L-iduronic or D-glucuronic acid sulfated at different sites, having a wide range of molecular weights, and are generally used as anticoagulants and antithrombotic compositions. Low-molecular weight heparins are heteregenous depolymerized products having a lesser degree of polymerization. The resulting biological properties are a function of polymer chain length and molecular weight distribution. In addition, the method of preparation of mucosal GAG's yield products with different end groups. One such end group is 2-5 anhydro-D-mannose, a result of enzymatic depolymerization.

The importance of this class of compounds in clinical medicine is based on its profound action on the coagulation system. Systemic coagulation is an integral part of many diseases and appears as a defense mechanism in trauma. Activation by the coagulation mechanism is a necessary part of the host's immune system. The coagulation system is not only involved in preventing blood loss following trauma, but clotting pathways may be involved in the pathology of allergy and inflammation. Deposits of fibrin surrounding cancer cells may also aid in the progression of growth of malignant tumors. There are multiple other properties of GAG's which are of potential interest as therapeutic agents.

The sequences in the linear GAG polymers that are responsive to the serine protease activity have been chemically synthesized. The natural glycosaminoglycans of interest as well as the synthetic analogs are made up of alternative units of L-iduronic and D-glucuronic acid: D-glucosamino units that are N-sulfated and N-acetylated. The 1-4 linked L-iduronic acids and the D-glucosamino acids have o-sulfate groups.

Although the therapeutic effects of these compounds is well documented, their use is limited because of their poor oral absorption. Injectable forms of these compounds exist and efforts have been made to provide an orally absorbable formulation of these compounds. An object of this invention is to provide compositions which are orally absorbable, and thus make them available for a broad spectrum of clinical conditions including cancer.

### SUMMARY OF THE INVENTION

This invention provides a composition comprising a depolymerized glycosaminoglycan or derivative thereof covalently linked to a bile acid.

This invention also provides a pharmaceutical composition comprising a depolymerized glycosaminoglycan or derivative thereof covalently linked to a bile acid and a pharmaceutically acceptable carrier.

The invention also provides an anticogulating treatment comprising administering to a subject in need thereof an effective anticoagulating amount of a depolymerized glycosaminoglycan or derivative thereof covalently linked to a bile acid and a pharmaceutically acceptable carrier.

### BRIEF DESCRIPTION OF THE FIGURES

FIG. 1 is a graph of in vitro anti-factor Xa Assay in human blood plasma for concentrations 2 ug/ml and 10 ug/ml. For each concentration, the first bar represents normal blood plasma; the second bar represents normal blood plasma and GAG; the third bar represents normal plasma and GAG derivative 1; and the fourth bar represents normal plasma and GAG derivative 2.

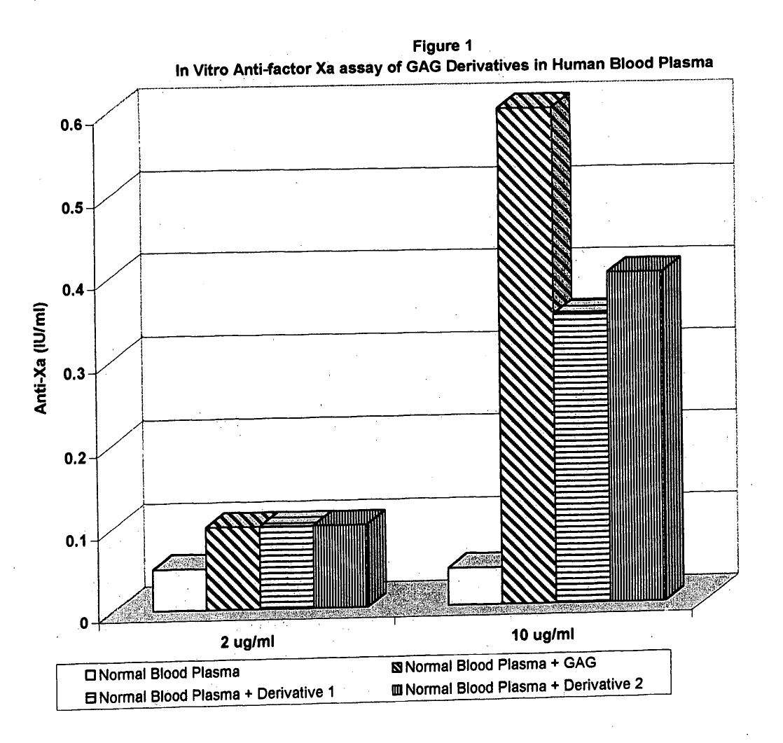

FIG. 2 is a graph of in vitro aPTT Assay of GAG in human blood plasma for concentrations 2 ug/ml and 10 ug/ml. For each concentration, the first bar represents normal blood plasma; the second bar represents normal blood plasma and GAG; the third bar represents normal plasma and GAG derivative 1; and the fourth bar represents normal plasma and GAG derivative 2.

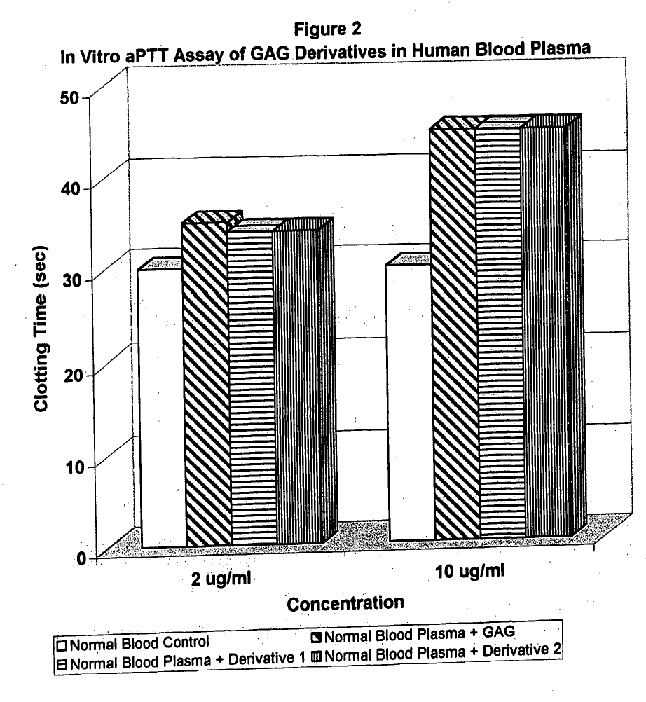

FIG. 3 is a graph of aPTT Assay at two hours following intestinal rejection. Each of the five columns of the four groups of rats represents one rat. The first bar represents the control (sham surgery); the second bar represents the GAG control; the third bar represents the GAG derivative 1; the fourth bar represents the GAG derivative 2; and the fifth bar represents the GAG derivative 3.

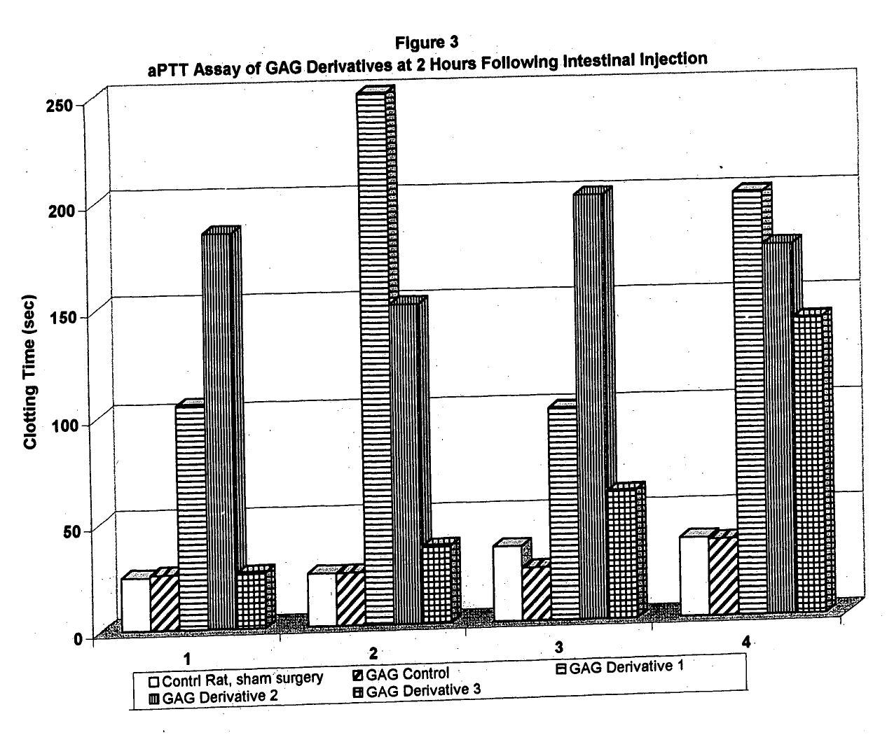

FIG. 4 is an NMR spectra of GAG derivative of cholic acid, GAG and GAG-deoxycholic acid. The peaks of the hexose protons are identified as well as the major doublet of the bile acid methyl group.

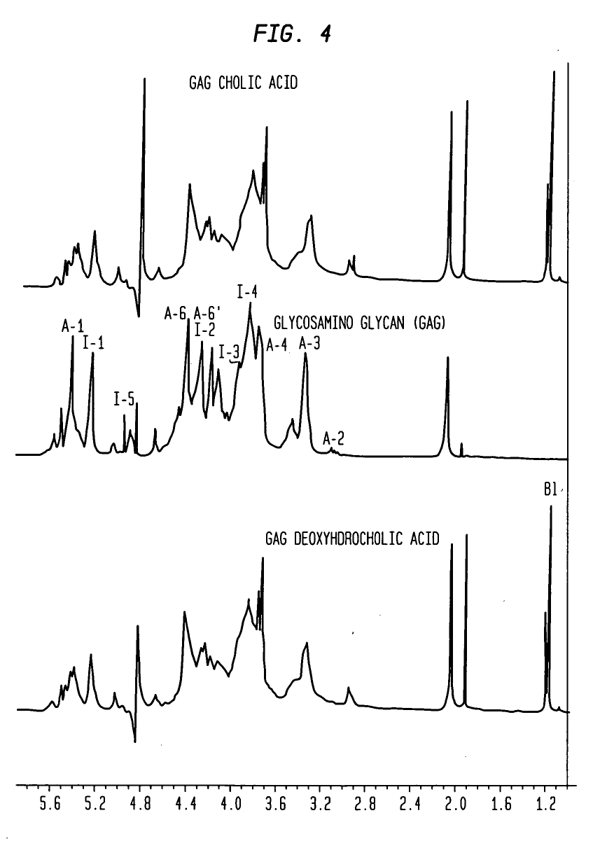

### DETAILED DESCRIPTION OF THE INVENTION

As stated above, the rationale for developing orally administrable glycosaminoglycans (GAG's) is based on the current and extensive use of this class of compounds that is only available in injectable form. In order to make glycosaminoglycans orally administrable, bile acid derivatives of GAG's have been prepared.

The bile acids are absorbed by ionic and non-ionic diffusion—mostly by passive ionic diffusion. The dehydroxy bile acids are more effectively absorbed as are the unconjugated bile acids. The bile acids are absorbed in the ileum by passive diffusion and by active transport. Structural variants in the bile acids influence absorption at various sites along the intestine.

Bile acids, their derivatives or analogs and bile pigments such as urobilin, urobilinogen and bilirubin that occur naturally, can be recognized by intestinal membranes as carriers. A family of derivatives, which can serve as absorbable carrier systems for glycosaminoglycans both natural and synthetic that are otherwise non-absorbable, has been developed.

Depolymerized GAGs can be prepared by enzymatic or free radical methods. These preparations can be bound at the carboxyl group of the bile acid. The GAGs prepared by either enzymatic depolymerization or free radical depolymerization can also be bound at the C3 position of the bile acid. Specific synthetically prepared GAGs (pentasaccharide to octasaccharide or higher polymers) can be bound at the carboxyl or at the C3 position of bile acids or analogs.

Using these endogenous chemical structures that are synthesized in vivo and recognized by intestinal membranes as carriers, we have developed a family of derivatives that can serve as absorbable carrier systems for glycosaminoglycans both natural and synthetic that are otherwise non-absorbable:
1) a depolymerized natural product (GAG) substituted at carboxyl of a bile acid using
   a.) a fractionated enzymatic product (tetramer to octamer), or
   b.) a fractionated free radical depolymerized product.
2) a depolymerized natural product (GAG) substituted at C3 position of a bile acid using
   a.) a fractionated enzymatic product (tetramer to octamer), or
   b.) a fractionated free radical depolymerized product.
3) a synthetically prepared analogs of GAG (pentasaccharide to octasaccharide)
   a.) a substituted at carboxyl of bile acid, or
   b.) a substituted at C-3 position of bile acid.

This invention provides a composition comprising a depolymerized glycosaminoglycan or derivative thereof covalently linked to a bile acid. The bile acid may comprise cholic acid, glychocholic acid, deoxycholic acid, dehydrocholic acid, chenodeoxycholic acid, or lithocholic acid. In one embodiment, the glycosaminoglycan are heparins. The glycosaminoglycan may be depolymerized by enzymatic or by free radical techniques.

In another embodiment of the invention, the depolymerized glycosaminoglycan, following exposure of the amino group is covalently linked to the carboxyl end of the bile acid. The N-sulfate group of the depolymerized N-desulfated glycosaminoglycan is covalently linked to the carboxyl end of the bile acid.

In a further embodiment, the depolymerized glycosaminoglycan is covalently linked to the C-3 position of the bile acid. The N-sulfate group of depolymerized glycosaminoglycan, following exposure of the amino group, is covalently linked to the C-3 position of the bile acid.

In yet another embodiment, the derivative of the depolymerized glycosaminoglycan comprises a synthetic analog of the glycosaminoglycan. The synthetic analog may comprise a pentasaccharide, a hexasaccharide, a heptasaccharide or an octasaccharide.

This invention also provides a pharmaceutical composition comprising a depolymerized glycosaminoglycan or derivative thereof covalently linked to a bile acid and a pharmaceutically acceptable carrier. In one embodiment, the depolymerized glycosaminoglycan of the pharmaceutical composition are heparins.

As used herein, “pharmaceutically acceptable carrier” means any of the various carriers known to those skilled in the art.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbonyl and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Finally, this invention provides a method for orally delivering a glycosaminoglycan to a subject comprising administering to the subject a pharmaceutically effective amount of one of the above-mentioned pharmaceutical compositions. In one embodiment, the glycosaminoglycan is heparin.

The invention also provides an anticoagulating treatment comprising administering to a subject in need thereof an effective anticoagulating amount of a depolymerized glycosaminoglycan or derivative thereof covalently linked to a bile acid and a pharmaceutically acceptable carrier.

Preparation of Modified Glycosaminoglycans

Commercially available crude product of digested porcine mucosa or fresh porcine mucosa from an abbatoir may be used as starting materials. Porcine intestinal mucosa may be enzymatically digested and the solids removed by centrifugation. The solubilized glycosaminoglycans containing solution is passed through a Dowex ion exchanger in the acid form and is eluted from the column with sodium chloride to produce a broad range of molecular weight GAGs. The GAGs of interest are purified from the mixture with cetyl-pyridinium chloride followed by salt and alcohol to further separate the undesirable carbohydrates. The products of this preliminary separation are subjected to enzymatic depolymerization according to the method first described by Pajza, N and Korn G. D., Biochem. Acta, vol. 20, page 596 (1956) or by a free radical method described by Cifonelli, Methods in Carbohydrate Chemistry, vol. 7, pages 139-141 (1976), using nitrous acid. The fragments produced by either enzymatic or free radical depolymerization after fractionation by column methods or by fractional precipitation produce unique species of GAG's with an ability to inhibit the coagulation process as for example Factor Xa inhibitors.

Enzymatic Depolymerization

Using flavobacteria digestion, the lower molecular weight GAG's are prepared by inoculating 12 grams of mucosal crude GAG with 6 grams of crude flavobacteria extract in 500 ml of 0.02 N phosphate buffer at 25° C. for 72 hours. The reaction is stopped by the addition of cold 20% trichloroacetic acid. The mixture is filtered and the filtrate is dialyzed for about 16 hours against three changes of distilled water in seamless cellulose dialyzer tubing with a 3500 D cutoff. The sodium salt of GAG is prepared by lyophylization of the mixture solution.

Free Radical Depolymerization

A relatively mild depolymerization method uses organic nitrite at low temperature which provides cleavage at specific linkages. This yields unaltered fragments which can be separated by a variety of methods including high pressure liquid chromatography, and alcohol-water precipitation. Free amino groups and other sulfamino groups are reactive sites and ester sulfates and N-acetyl groups and otherwise easily hydrolysable linkages survive free radical depolymerization.

A 0.1 M solution of nitrous acid reagent is prepared by mixing a 3.5% solution of sodium nitrate (10 ml) with 1,2 dimethoxyethane (20 ml) and 2M hydrochloric acid (5 ml) and cooled to −20° C. The solution is used within a few hours of preparation. The free acid form of the glycosaminoglycan (250 mg) is used for the depolymerization. The reaction is maintained for 1 hour with mixing and the reaction is inhibited by adding 12% ammonium sulfamate (final concentration). The reaction mixture is eluted with 0.2 M sodium acetate in 10% ethanol following neutralization by gel filtration on SEPHADEX G-25. The first major fraction eluted has a molecular weight of 3000 Da and the second major fraction is a tetrasaccharide.

Synthesis of Bile Acid Derivative of GAG at Carboxyl Group of Bile Acid

The products of degradation of mucosal GAG by either enzymatic or free radical methods followed by fractionation will have N-sulfate groups that are the site of derivitization in the bile acids. In the process of derivatization, the sulfate is removed to expose the reactive amino group. The sodium salt of the GAG is converted to the free acid on a DOWEX SO₃H column and rapidly converted to the pyridium salt to protect the COOH group.

Synthesis of Bile Acid Derivatives of GAG at the C3 Position of Bile Acid

The bile acid ester reacted with allyl bromide in the presence of pyridine gives the allyl derivative which is reduced with borohydride and oxidized with hydrogen peroxide in an alkaline system. This gives the 3-hydroxy derivative methyl sulfonyl chloride. Pyridine addition is followed by deamination with sodium azide followed by reduction with hydrogen gas using a palladium catalyst in DMSO. This provides a carboxyl group in the C-3 position of the bile acid that is now available for binding the GAG at the amino position using thiophosgene.

The following examples are illustrative of the practice of the invention and should not be considered limiting.

### EXAMPLE 1

One half gram of pyridine-GAG is dissolved in 23 ml of DMSO in 3 ml H₂O and maintained at a pH of 4.75. Cholic acid (0.15 gm) is dissolved in 20 ml of DMSO in 3 ml of H₂O to this added 0.5 gm of EDC (1 ethyl C-3-C₃-dimethylene propyl) carbodiamide and the mixture added to pyridine GAG with constant stirring, maintaining the pH at 4.75 at 25° C. for two hours.

The reaction products are dialyzed against 2 liters of water with three changes using seamless tubing with a 3500 D cutoff. The precipitate that forms is removed by filtration. The filtrate is lyophyllized and the white powder is dissolved in 10 ml of sodium acetate (6.8%) and the pH adjusted to 7.8. Three volumes of ethyl alcohol are added to the solution and the precipitate is recovered by centrifugation at 2500 RPM and washed with alcohol. The dried precipitate is then dissolved in water and lyophyllized. The yield of final product is 70%.

### EXAMPLE 2

One gram of pyridine-GAG in 20 ml DMSO containing 3 ml H₂O is maintained at pH 4.75. The DMSO soluble dehydrocholic acid (1 gm) was activated by one gram EDC in DMSO for 2 hours at room temperature. The pyridine GAG was added, the mixture maintained at a pH of 4.75 overnight, and then dialyzed, centrifuged and finally lyophylized. The product was dissolved in 10 ml of sodium acetate and the pH adjusted to 7.8 and then treated as in example 1.

### EXAMPLE 3

One gram of pyridine-GAG was dissolved 20 ml DMSO containing 3 ml H₂O and the pH maintained at 4.75. 0.25 gm of glycholic acid was activated in 20 ml DMSO containing 3 ml H₂O. By adding 0.5 gm EDC the mixture was added to pyridine GAG with constant stirring while maintaining pH at 4.75 at 25° for 2 hours. The procedure in Example 1 was followed.

In Vivo Bioassays of GAG (Carboxyl Derivative) Bile Acid

Assays were performed on 200 gm Sprague Dawley Rats that fasted for 24 hours. Rats were initially anesthetized for abdominal surgery. Test compounds were injected directly into a closed bowel loop produced by ligation at the duodenium and at the junction, using a 30 gauge needle containing 2 ml of a 5 mg/ml solution. At two hours venous or cardiac blood samples were drawn for assay.

Assay of GAG and GAG-Bile Acid Derivatives

By anti Xa Method

The Stago Statchron Kit # 00906 using a Dade Behring BCS instrument calibrated with Lovenox diluted in pooled plasma was used. In this asssay paranitroamide released is measured at 405 nm and is inversely proportional to the amount of GAG present in the plasma sample.

By aPTT Assay

The clotting system contains purified phospholipids (porcine and chicken), micronized silica-activator, buffer, stabilizer and preservative. Clotting time is measured with the Dade Behring BCS instrument. Assays were performed by Blood Center of Southwestern Wisconsin. The results were as follows:

In Vitro APTT Response versus Concentration of Oral Heparin Derivative

| 40 | 50/50/50 | 75/75/60 | 125/125/100 | 140/140 | 200/200 |
|---|---|---|---|---|---|
| 0 | 10 | 20 | 30 | 40 | 50 |

### Concentration (ug/mL)

### Triplicate Readings, Digital and Fibrometer Measurements

Proton NMR Spectroscopy of GAG and Bile Acid Derivatives

Deuterated water solutions of the compounds were examined at 360 megahertz in an NMR spectrophotometer. Signals were measured at displacements of TMS (sodium 3-trimethyl silyl proprionate 2, 2, 3, 3 d₄). Characteristic peaks were assigned to protons at the one position of glucosamino-N sulfated unit at 5.4 ppm, the five position of 2-O-sulfated iduronic acid at 5.2 ppm and the 0.5 position of 2-O-sulfated iduronic acid at 4.8 ppm (GATTI et al., Macromolecules Vol. 12, pages 1001-1007, 1979). The references cited herein are incorporated by reference.

What is claimed is:

1. A composition comprising a depolymerized glycosaminoglycan or derivative thereof covalently linked to a bile acid.

2. The composition according to claim 1, wherein the bile acid is cholic acid, glycholic acid, deoxycholic acid, dehydroxycholic acid, chenodeoxycholic acid or lithocholic acid.

3. The composition according to claim 1, wherein the glycosaminoglycan is a low molecular weight heparin.

4. The composition of claim 1, wherein the depolymerized glycosaminoglycan is depolymerized by enzymatic depolymerization.

5. The composition of claim 1, wherein the depolymerized glycosaminoglycan is depolymerized by free radical depolymerization.

6. The composition of claim 1, wherein the depolymerized glycosaminoglycan comprises an N-sulfate group.

7. The composition of claim 1, wherein the depolymerized glycosaminoglycan is covalently linked to a carboxyl group at one end of the bile acid.

8. The composition of claim 6, wherein the N-sulfate group of the depolymerized glycosaminoglycan following N-desulfation is covalently linked to a carboxyl group at one end of the bile acid.

9. The composition of claim 1, wherein the depolymerized glycosaminoglycan is covalently linked to at C-3 position of the bile acid.

10. The composition of claim 6, wherein the N-sulfate group of depolymerized glycosaminoglycan following N-desulfation is covalently linked to the C-3 position of the bile acid.

11. The composition of claim 1, wherein the derivative of the depolymerized glycosaminoglycan comprises a synthetic analog of the glycosaminoglycan with a free amino group.

12. The composition of claim 11, wherein the synthetic analog comprises a pentasaccharide, a hexasaccharide, a heptasaccharide or an octasaccharide.

13. A pharmaceutical composition comprising a composition comprising a depolymerized glycosaminoglycan or derivative thereof covalently linked to a bile acid and a pharmaceutically acceptable carrier.

14. The pharmaceutical composition of claim 13, wherein the depolymerized glycosaminoglycan is heparin or a low molecular weight heparin, or a derivative thereof.

15. A method for orally delivering a glycosaminoglycan to a subject comprising administering to the subject a pharmaceutically effective amount of the pharmaceutical composition of claim 13.

16. A method for orally delivering glycosaminoglycan to a subject comprising administering to the subject an anti-coagulating treatment comprising administering to a subject in need thereof an effective anticoagulating amount of a depolymerized glycosaminoglycan or derivative thereof covalently linked to a bile acid and a pharmaceutically acceptable carrier.
